(19) United States
(12) Patent Application Publication
(10) Pub. No.: US 2013/0337473 A1
(43) Pub. Date: Dec. 19, 2013

(54) CELL IDENTIFICATION SYSTEMS AND METHODS USING FUNCTIONALIZED MICROPALLET ARRAYS

(71) Applicant: The Regents of the University of California, (US)

(72) Inventors: Edward L. Nelson, Irvine, CA (US); Trisha M. Westerhof, Irvine, CA (US); Mark Bachman, Oakland, CA (US); Guann-Pyng Li, Oakland, CA (US)

(73) Assignee: The Regents of the University of California, Oakland, CA (US)

(21) Appl. No.: 13/888,299

(22) Filed: May 6, 2013

Related U.S. Application Data
(60) Provisional application No. 61/643,146, filed on May 4, 2012.

(51) Int. Cl. G01N 33/574

(52) U.S. Cl. CPC G01N 33/574
USPC 435/7.23; 435/7.1

(57) ABSTRACT

Cell identification systems and methods using micropallet arrays are disclosed. According to one embodiment, a cell identification strategy involves detecting the expression patterns of markers that are expressed on cells to uniquely identify different cell populations within heterogeneous mixtures of cells. The cell markers may be on the cell surface or intracellular in location. The cell markers are interrogated using monoclonal antibodies (mAbs) that are directly conjugated to flourophores. The mAb-flourophore conjugates are used to detect the presence or absence and relative level of expression of each of the cell markers using laser scanning confocal microscopy.

The present application claims the benefit of and priority to U.S. Provisional Application No. 61/643,146 titled “CELL IDENTIFICATION SYSTEMS AND METHODS USING MICROPALLET ARRAYS,” filed on May 4, 2012, which is hereby incorporated by reference in its entirety.

### FIELD

The embodiments provided herein relate generally to micropallet arrays used for the separation and collection and single adherent cells from within a cellular population, and more particularly to systems and methods that facilitate the use of a micropallet array platform in the identification of cells within a heterogeneous population by the incorporation of a multicolor immunoflourescent confocal imaging strategy.

### BACKGROUND

An increasingly widespread requirement in biomedical research is the positive selection of single cells from populations of cells. Biologists have an increasing number and variety of tools with which to analyze single cells at the molecular level, including microscopy, PCR, patch-clamp, and microanalytical chemical separations, yet have very limited tools to enable the selection of a single cell from a large cellular population.

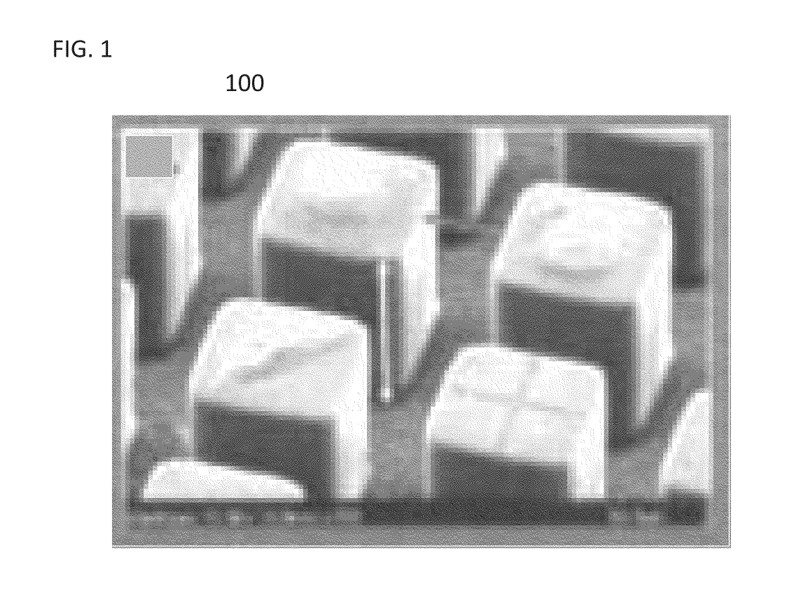

FIG. 1 illustrates an SEM micrograph 100 of a micropallet array. Micropallet arrays, the fabrication of which has been previously disclosed in U.S. Published Patent Application No. 2007-0292312, are composed of many individual, releasable polymer pedestals (micropallets) that isolate adherent cells in culture. They can be released from the substrate (glass or otherwise) using a laser or otherwise. Magnetically responsive, ferromagnetic micropallets, which have been previously disclosed in U.S. Pat. Nos. 7,659,954 and 7,951,580, are collectable using magnetically-based systems. In use, the micropallets or microstructures can be released from the substrate by any means including, but not limited to, laser-based release or direct-contact mechanical disruption of the attachment to the underlying substrate, after which they are collected using a magnetic collection probe based on magnets.

Once released, the individual micropallets are collectable such that any cell or cells adhered to a single micropallet can be isolated, selected, and collected from a larger cellular population. The process has been shown to cause minimal perturbation to the released and recovered cells and the cells remain viable and capable of expanding into clonal populations (derived from a single cell) after recovery.

While this system is appropriate for collecting single cell samples within pure populations of a single cell type, the user is unable to effectively apply this collection strategy to a heterogeneous cell population plated on a micropallet array, because of the platform's inability to uniquely identify cell types within mixed populations of cells.

Although several strategies exist to selectively identify and collect cells from mixed populations, each has significant drawbacks for the analysis of primary adherent tumor cells. Fluorescence-activated cell sorting (FACS) requires large numbers of cells that have been subjected to enzymatic or mechanical tissue disruption. Laser capture microdissection (LCM) can collect single cells or small groups of selected cells or (non-viable) from fixed or frozen tissue sections. Live cell LCM protocols have been reported, but are uniformly inefficient, low throughput and not suitable for isolating significant numbers of single, living cells. Recently, live cell microarray technologies based upon ligand-receptor interactions have been reported, but these are critically dependent upon single, unique discriminating interactions for each cell type. These technologies are not readily applicable to the identification and recovery of rare cells that require multi-parameter detection and are poorly suited to evaluating multiple discrete cell populations within a complex sample. Although multicolor imaging, albeit not six channel, has been used extensively in histologic sections and whole mount preparations to identify cells expressing one or more markers, as noted above, these strategies do not permit the recovery of single cells in a manner amenable to analyses of cells from several subsets from a single sample.

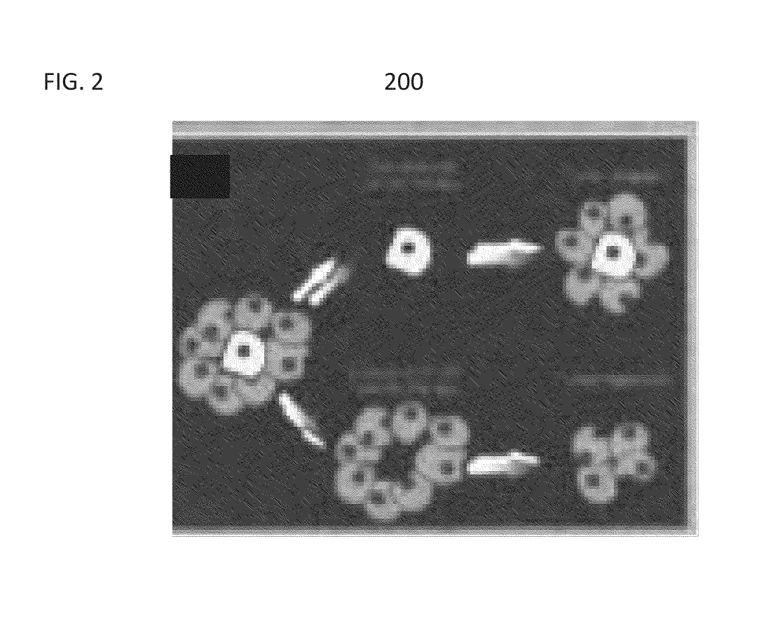

FIG. 2 illustrates a cancer stem cell hypothesis 200. Breast cancer patients, even those with histologically identical tumors, experience substantial variability in clinical behavior and response to treatment. Differences in the profile of tumors, such as the proportion of cancer stem cells (CSCs), may be a source of this variability. Cell subsets of interest can be identified by their expression of a panel of cell surface markers (see Table 1).

TABLE 1
Cell Surface Marker Expressions of Human Primary
Breast Tumor Cell Subsets.

| | ESA | CD44 | CD10 | CD24 | CD133 | CD309 |
|---|---|---|---|---|---|---|
| Epithelial Tumor Cells | + | + | − | + | − | − |
| Mammary Tumor Stem | + | + | − | − | − | − |
| Cells | | | | | | |
| Myoepithelial Cells | − | +/− | + | − | − | − |
| Endothelial Progenitor | − | − | − | + | + | + |
| Cells | | | | | | |

In some applications, for example, the cancer stem cell hypothesis 200, the ability to analyze these rare cell subsets could lead to the development of more efficacious cancer therapies that directly target these cells.

### SUMMARY

Cell identification systems and methods using micropallet arrays are disclosed. According to one embodiment, a cell identification strategy involves detecting the expression patterns of markers that are expressed on cells to uniquely identify different cell populations within heterogeneous mixtures of cells. The cell markers may be on the cell surface or intracellular in location. The cell markers are interrogated using monoclonal antibodies (mAbs) that are directly conjugated to flourophores. The mAb-flourophore conjugates are used to detect the presence or absence and relative level of expression of each of the cell markers using laser scanning confocal microscopy.

In another embodiment, a kit for identifying cells is provided that includes an array of micro-pallets releasably coupled to a substrate, and first and second groups of mAb-fluorophores. The first and second groups of mAb-fluorophores comprising differing cell markers identifiable by differing fluorophore emission spectra

The systems, methods, features and advantages of the invention will be or will become apparent to one with skill in the art upon examination of the following figures and detailed description. It is intended that all such additional methods, features and advantages be included within this description, be within the scope of the invention, and be protected by the accompanying claims. It is also intended that the invention is not limited to require the details of the example embodiments.

### BRIEF DESCRIPTION

The accompanying drawings, which are included as part of the present specification, illustrate the presently preferred embodiment and, together with the general description given above and the detailed description of the preferred embodiment given below, serve to explain and teach the principles of the present invention.

FIG. 1 illustrates an SEM micrograph of a micropallet array.

FIG. 2 illustrates a cancer stem cell hypothesis

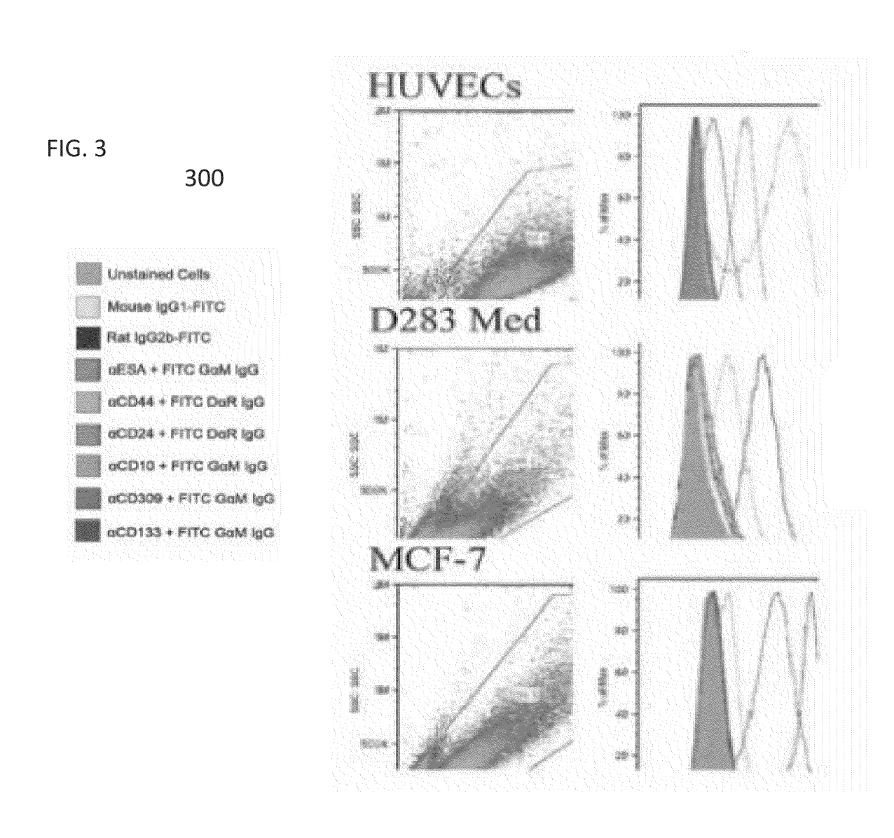

FIG. 3 illustrates an exemplary multicolor imaging strategy, according to one embodiment.

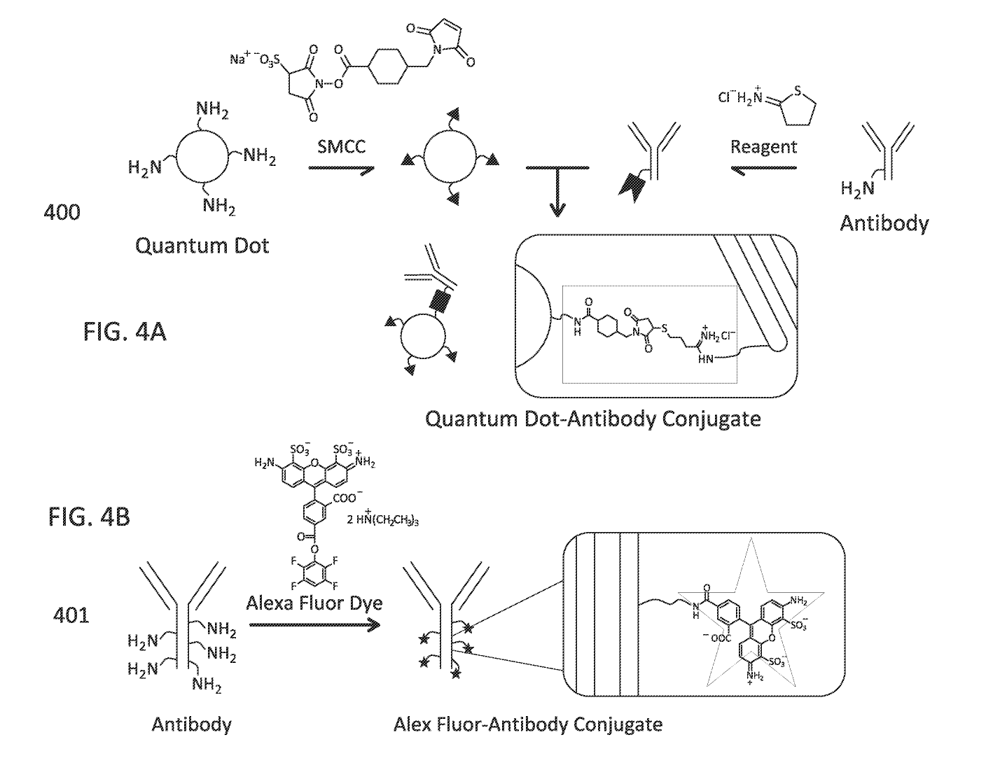

FIG. 4A illustrates an exemplary Qdot-mAb conjugation scheme used to couple monoclonal antibodies to respective fluorophores.

FIG. 4B illustrates an exemplary Alexa Fluor-mAb conjugation scheme used to couple monoclonal antibodies to respective fluorophores.

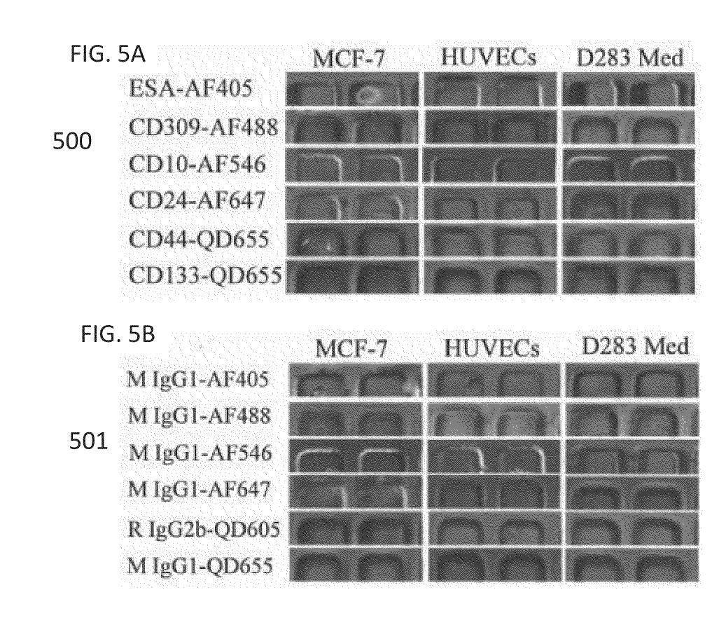

FIG. 5A illustrates exemplary immunoflourescent detection of single cell surface markers using fluorophore conjugated monoclonal antibodies, according to one embodiment.

FIG. 5B illustrates exemplary conjugation of mouse IgG1 and rat IgG2b isotype antibodies to respective dyes as controls, according to one embodiment.

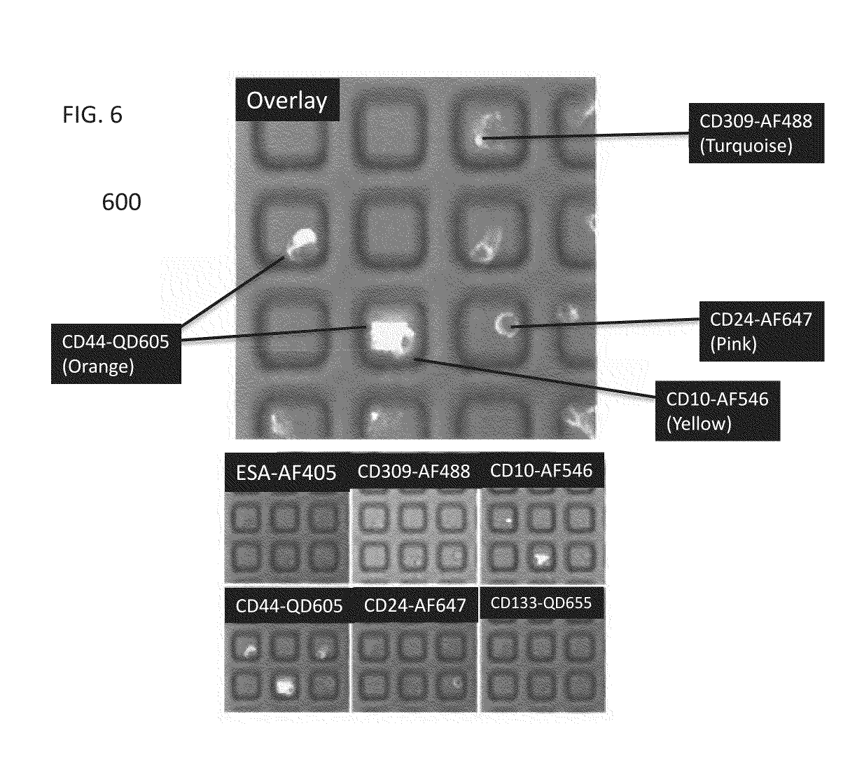

FIG. 6 illustrates exemplary multicolor immunoflourescent detection of cell surface markers, according to one embodiment.

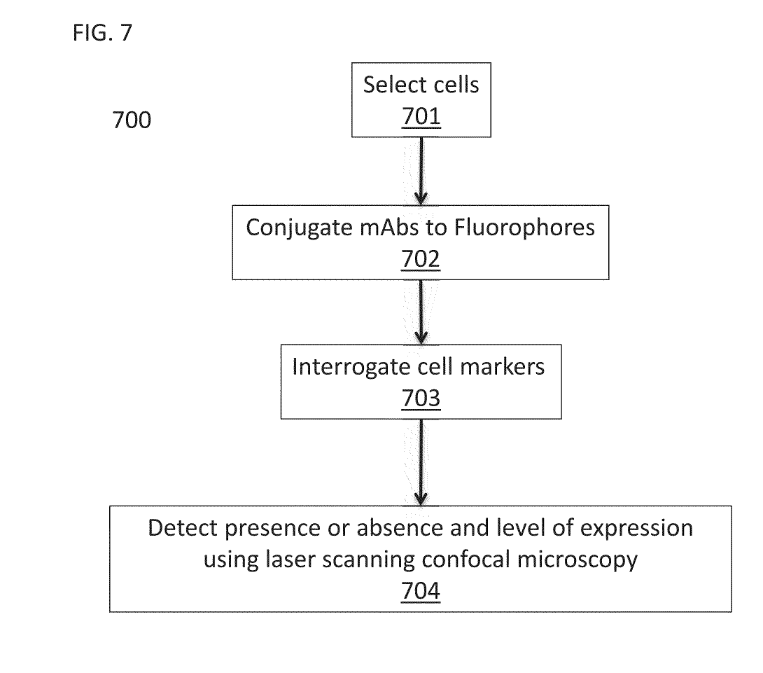

FIG. 7 illustrates an exemplary process for use with the present system, according to one embodiment.

It should be noted that the figures are not necessarily drawn to scale and that elements of similar structures or functions are generally represented by like reference numerals for illustrative purposes throughout the figures. It also should be noted that the figures are only intended to facilitate the description of the various embodiments described herein. The figures do not necessarily describe every aspect of the teachings disclosed herein and do not limit the scope of the claims.

### DETAILED DESCRIPTION

The embodiments described herein are directed to a new method and system for identifying heterogeneous populations of cells to be incorporated into the existing micropallet array platform. The cell identification strategy involves detecting the expression patterns of markers that are expressed on cells to uniquely identify different cell populations within heterogeneous mixtures of cells. The cell markers may be on the cell surface or intracellular in location. The cell markers are interrogated using monoclonal antibodies (mAbs) that are directly conjugated to flourophores. The mAb-flourophore conjugates are used to detect the presence or absence and relative level of expression of each of the cell markers using laser scanning confocal microscopy. By using predetermined combinations of mAb-flourophores, it is possible to identify and differentiate between a variety of cell subsets that comprise complex tissues, for example neoplastic tumors. For the exemplary purposes only, the embodiments discussed herein focus on the identification strategy for cellular subsets comprising breast tumors, putative cancer stem cells, endothelial progenitor cells, myoepithelial cells, epithelial tumor cells, amongst others. The development of the multicolor immunofloursecent imaging strategy enables the application of the micropallet array technology to the identification, enumeration, and analyses of single adherent cells with defined phenotypes from complex samples.

The present immunofluorescent laser scanning confocal imaging strategy interrogates multiple cell surface markers whose expression or lack thereof uniquely identifies breast tumor cell subsets of interest; i.e., cancer stem cells, endothelial progenitor cells, etc., which enables the identification of unique cells within a heterogeneous population, and cannot be achieved with conventional cell sorting and identification methodologies, such as flow cytometry.

For proof of principle, a total of 3 cell lines, MCF-7, Human Umbilical Vein Endothelial Cells (HUVECs), and D283 Med, were selected based on their expression of the desired cell surface markers, and confirmed via flow cytometry. Purified monoclonal antibodies (mAbs) specific for each surface marker of interest were tested against each cell line to confirm their antigen reactivity using appropriate FITC labeled secondary IgG antibodies (see FIG. 3). These cells lines collectively express the panel of surface markers (Table 2) on primary cell subsets of interest in breast tumors. There is no single marker that uniquely identifies one cell type from another, necessitating the need for a panel of surface markers in order to uniquely identify the cell type.

FIG. 3 illustrates an exemplary multicolor imaging strategy 300, according to one embodiment. Cell lines that collectively express all the cell surface markers necessary to identify primary breast tumor cell subsets were used to develop the multicolor imaging strategy. The expression pattern of each marker, as validated via flow cytometry (a) is depicted as either expressed (+) or not expressed (−) in Table 2.

TABLE 2
Cell Surface Marker Expression of Control Cell Lines

| | ESA | CD44 | CD10 | CD24 | CD133 | CD309 |
|---|---|---|---|---|---|---|
| MCF-7 | + | + | − | + | +/−* | − |
| HUVECs | − | + | + | − | − | + |
| D283 Med | − | + | − | − | + | − |

*Asmall subset of MCF-7 cells have been reported to express CD133 and exhibit cancer stem cell properties.

FIG. 4A illustrates an exemplary Qdot-mAb conjugation scheme 400 used to couple monoclonal antibodies to respective fluorophores. FIG. 4B illustrates an exemplary Alexa Fluor-mAb conjugation scheme 401 used to couple monoclonal antibodies to respective fluorophores.

To interrogate the surface markers using laser scanning confocal microscopy, the flow cytometry validated purified mAbs were conjugated directly to Quantum dots (QD 400, FIG. 4A), and AlexaFluor dyes (AF 401, FIG. 4B) using established cross-linking chemistries. Covalent fluorophore-conjugated mAbs (primary labeling) vs. the use of secondary fluorophore reagents minimized the possibility of cross-reactivity and non-specific binding of secondary reagents within a given sample. In order to detect the expression of all the markers in a single sample, each fluorophore was paired with a mAb such that the brightest fluorophores were paired with mAbs specific for lowly expressed surface markers and vice versa (Table 3).

TABLE 3
mAb Fluophore conjugation schemes.

| mAb | Fluorophore |
|---|---|
| ESA | Alexa Fluor 405 |
| CD44 | Quantum Dot 605 |
| CD10 | Alexa Fluor 546 |
| CD24 | Alexa Fluor 647 |
| CD133 | Quantum Dot 655 |
| CD309 | Alexa Fluor 488 |

The resulting mAb-fluorophores were then used to stain and image each cell line (see FIG. 5A 500). Isotype antibodies were also conjugated to each fluorophore for appropriate negative controls (see FIG. 5B 501). FIG. 5A illustrates exemplary immunoflourescent detection of single cell surface markers using fluorophore conjugated monoclonal antibodies, according to one embodiment. FIG. 5B illustrates exemplary conjugation of mouse IgG1 and rat IgG2b isotype antibodies to respective dyes as controls, according to one embodiment.

Mixtures of the 3 cell lines were also stained and imaged to demonstrate the identification of each cell population based on their expression pattern of the 6 cell surface markers (see FIG. 6600). FIG. 6 illustrates exemplary multicolor immunoflourescent detection of cell surface markers, according to one embodiment.

FIG. 7 illustrates an exemplary cell identification strategy 700 for use with the present system, according to one embodiment. The cell identification strategy 700 includes detecting the expression patterns of markers that are expressed on cells to uniquely identify different cell populations within heterogeneous mixtures of cells. Cells are selected based on their expression of desired cell surface markers 701. The cell markers may be on the cell surface or intracellular in location. The cell markers are interrogated 703 using the monoclonal antibodies (mAbs) that are directly conjugated 702 to fluorophores (the mAb-fluorophores). The mAb-fluorophore conjugates are used to detect the presence or absence and relative level of expression of each of the cell markers using laser scanning confocal microscopy 704. By using carefully selected combinations of mAb-fluorophores, it is possible to identify and differentiate between a variety of cell subsets that comprise complex tissues, for example neoplastic tumors.

The present system significantly advances the technique to detect various cell types of interest based on their expression pattern of surface markers to functionalize the MPA platform towards analyzing human tumor specimens. The employment of this MPA technology with the presented multicolor immunofluorescent confocal imaging strategy will allow for future use in the isolation and study of single cells comprising human primary breast tumor specimens.

In the foregoing specification, the invention has been described with reference to specific embodiments thereof. It will, however, be evident that various modifications and changes may be made thereto without departing from the broader spirit and scope of the invention. For example, the reader is to understand that the specific ordering and combination of process actions shown in the process flow diagrams described herein is merely illustrative, unless otherwise stated, and the invention can be performed using different or additional process actions, or a different combination or ordering of process actions. As another example, each feature of one embodiment can be mixed and matched with other features shown in other embodiments. Features and processes known to those of ordinary skill may similarly be incorporated as desired. Additionally and obviously, features may be added or subtracted as desired. Accordingly, the invention is not to be restricted.

Cell identification systems and methods using micropallet arrays have been disclosed. It is understood that the embodiments described herein are for the purpose of elucidation and should not be considered limiting the subject matter of the disclosure. Various modifications, uses, substitutions, combinations, improvements, methods of productions without departing from the scope or spirit of the present invention would be evident to a person skilled in the art.

What is claimed is:

1. A method for identifying cells, comprising:
staining a plurality of cells with a plurality of mAb-fluorophores comprising monoclonal antibodies (mAbs) with a predetermined cell marker conjugated to fluorophores having a predetermined spectral emission, wherein individual ones of the plurality of mAb-fluorophores having spectral emissions differing from other individual ones of the plurality of mAb-fluorophores;
adhering individual ones of the plurality of cells to individual pallets in an array of micro-pallets releasably coupled to a substrate;
identifying individual cells of the plurality of cells based on a cell marker; and
releasing the pallet to which the identified cell is adhered from the substrate.

2. The method of claim 1, wherein the step of identifying individual cells includes detecting the presence or absence of a cell marker and a relative level of expression of the cell marker by interrogating the mAb-fluorophores using laser scanning confocal microscopy.

3. The method of claim 2, wherein the detected presence, absence, and relative level of expression of the cell marker are used to identify the cells.

4. The method of claim 1, wherein the cells comprise tumor cells.

5. The method of claim 1, wherein the fluorophores are selected from the group consisting of Alexa Fluors and Quantum Dots.

6. The method of claim 1, wherein the mAbs are selected from the group consisting of ESA, CD44, CD10, CD24, CD133, and CD309.

7. The method of claim 3, wherein the tumor cells comprise cellular subsets including one or more of breast tumor cells, putative cancer stem cells, endothelial progenitor cells, myoepithelial cells, and epithelial tumor cells.

8. A kit for identifying cells, comprising:
an array of micro-pallets wherein individual pallets in the array of micro-pallets are releasably coupled to a substrate wherein the substrate remains intact upon release of an individual pattet;
a first plurality of mAb-fluorophores, wherein individual ones of the first plurality of mAb-fluorophores having a first cell marker identifiable by a first fluorophore emission spectra; and
a second plurality of mAb-fluorophores, wherein individual ones of the second plurality of mAb-fluorophores having a second cell marker identifiable by a second fluorophore emission spectra, wherein the second fluorophore emission spectra differs from the first fluorophore emission spectra.

9. The kit of claim 8, wherein individual ones of the a first plurality of mAb-fluorophores comprise a monoclonal antibody (mAbs) having the first predetermined cell maker conjugated with a fluorophore having the first fluorophore emission spectra when excited by an excitation laser, and wherein individual ones of the second plurality of mAb-fluorophores comprise a monoclonal antibody (mAbs) having a second predetermined cell maker conjugated with a fluorophore having the second fluorophore emission spectra when excited by an excitation lasers.

10. The kit of claim 9, wherein fluorophores of the first and second plurality of mAb-fluorophores are selected from a group consisting of Alexa Fluors and Quantum Dots.

11. The method of claim 10, wherein mAbs of the first and second plurality of mAb-fluorophores are selected from the group consisting of ESA, CD44, CD10, CD24, CD133, CD34, CD184, and CD309.
